(19) United States
(12) Patent Application Publication
(10) Pub. No.: US 2020/0113461 A1
(43) Pub. Date: Apr. 16, 2020

(54) NON-INVASIVE METHOD FOR DETERMINING INTRACRANIAL PRESSURE USING THE BIOELECTRICAL ACTIVITY OF THE BRAIN

(71) Applicant: FUNDACION PARA LA INVESTIGACION BIOMEDICA DEL HOSPITAL UNIVERSITARIO DE LA PRINCESA, Madrid (ES)

(72) Inventors: Ancor SANZ GARCIA, Madrid (ES); Jesús PASTOR GÓMEZ, Madrid (ES); Lorena Carolina VEGA ZELAYA, Madrid (ES); María del Carmen TORRECILLA LOPEZ, Madrid (ES); Maria Gema VEGA GONZALEZ, Madrid (ES); Fernando MONASTERIO CHICHARRO, Madrid (ES); Rafael GARCIA DE SOLA, Madrid (ES); Paloma PULIDO RIVAS, Madrid (ES); Cristina Virginia TORRES DIAS, Madrid (ES)

(21) Appl. No.: 16/626,272

(22) PCT Filed: Apr. 30, 2018
(86) PCT No.: PCT/ES2018/070334

§ 371 Date: Dec. 23, 2019

(30) Foreign Application Priority Data
Jul. 18, 2017 (ES) P201730943

(51) Int. Cl. A61B 5/03; A61B 5/048; G16H 10/60; G16H 50/20

(52) U.S. Cl. CPC A61B 5/031; G16H 50/20; G16H 10/60; A61B 5/048

(57) ABSTRACT

The present invention relates to a non-invasive method for determining changes in intracranial pressure using data obtained from an electroencephalogram recorded for a patient comprising:
a) determining the values of the spectral analysis and network variables for the electroencephalogram,
b) determining the endogenous variable X for a transfer function the exogenous variables of which are the values for the variables obtained in step a),
   whereby changes in the endogenous variable X are indicative of changes in the value of the intracranial pressure; the present invention also relates to a device for carrying out the method of the present invention.

### FIELD OF THE INVENTION

The present invention falls within the general field of biomedicine, and in particular relates to a non-invasive method and system for determining changes in intracranial pressure using data obtained from an electroencephalogram recorded for a patient.

### STATE OF THE ART

Continuous multimodal monitoring of neurocritical patients is a widespread practice nowadays in most intensive care units (ICUs). Both neurosurgeons and intensive care specialists have a large arsenal of techniques that enable them to continuously monitor critical variables in patients with a wide variety of pathologies. Cases involving traumatic brain injury (TBI) and subarachnoid haemorrhage (SAH) are of particular interest. In these patients, it is useful to monitor intracranial pressure (ICP), cerebral perfusion pressure (CPP), tissue oxygen pressure (PtiO2) and electrical activity of the brain by using electroencephalography (EEG), among many other variables. The value of using it to identify, prevent and treat secondary injuries which can worsen the primary pathology of the patient is certainly indisputable nowadays [Le Roux, P., Menon, D. K., Citerio, G., Vespa, P., Bader, M. K., Brophy, G. M., . . . & Badjatia, N. (2014). Consensus summary statement of the international multidisciplinary consensus conference on multimodality monitoring in neurocritical care. Neurocritical care, 21(2), 1-26]. Multimodal monitoring is helpful in those cases in which the clinical examination of the patient may be impossible due to the effects of sedoanalgesia or in those cases in which the patient is in an altered state of consciousness, such as when they are in a coma. Moreover, the information provided by the monitoring equipment usually precedes the clinical information, for which reason changes in the monitored variables help detect changes in the underlying physiological processes and therefore predict any change in the clinical condition.

One of the variables that must be monitored in any neurocritical patient is the ICP, the measurement of which is highly invasive since it is carried out by means of a sensor which measures the pressure of the cerebrospinal fluid either intraparenchymally or intraventricularly, and therefore requires surgery for the placement thereof.

Therefore, there is a need to provide a non-invasive alternative in order to determine the risk associated with an increase in intracranial pressure via a non-invasive method.

### DESCRIPTION OF THE INVENTION

The present invention solves the problems described in the state of the art since it refers to a non-invasive method and system for determining intracranial pressure in a subject. Thus, in a first aspect, the present invention relates to a non-invasive method for determining changes in intracranial pressure using data from an electroencephalogram recorded for a patient (hereinafter, method of the present invention) comprising:
a) determining the values of the spectral analysis and network variables for the electroencephalogram,
b) determining the endogenous variable X for a transfer function the exogenous variables of which are the values for the variables obtained in step a),
   whereby changes in the endogenous variable X are indicative of changes in the value of the intracranial pressure.

In a particular embodiment of the present invention, the spectral analysis variables for the electroencephalogram determined in step a) are selected from: bands lower than delta (<1 Hz), delta (1-4 Hz), theta (4-7 Hz), alpha (8-12 Hz), beta (12-30 Hz), gamma (30-100 Hz), bands greater than gamma (>100 Hz), spectral entropy of all frequencies. In a more particular embodiment, at least two of the spectral analysis variables are determined in step a) of the method of the present invention.

In one particular embodiment of the present invention, the network analysis variables for the electroencephalogram determined in step a) are selected from among the variables obtained from the synchronisation between the different electrodes of the electroencephalogram: density of links, average clustering coefficient, mean path length and degree of each electrode of the encephalogram or node. In a more particular embodiment, at least two of the network analysis variables are determined in step a) of the method of the present invention.

In the present invention, channel and node refer to the same element, namely the data obtained from an encephalogram, but from different perspectives. Therefore, channel refers to the signal received through each electrode of the electroencephalogram. When the network analysis is performed, instead of a channel it is called a node and it refers to each of the points making up the network.

In a more particular embodiment of the present invention, the value of the spectral analysis and network variables of step a) is determined by means of the following steps:
I. calculating the power spectrum in the neurophysiological records from each electrode or channel, by means of the Fourier transform;
II. calculating the relative power of each of the characteristic frequency bands of the electroencephalogram;
III. calculating the value of entropy and spectral entropy;
IV. calculating synchronisation measurements between all pairs of neurophysiological records using Pearson correlation, Mutual Information and Phase Synchronisation;
V. calculating the value of the density of links, average clustering coefficient, mean path length and degree of each node;
VI. estimating all the previous measurements in consecutive time windows.

In a particular embodiment, the method of the present invention optionally comprises determining variables associated with the heart rate variability of an electrocardiogram recorded for a patient.

In another particular embodiment, the method of the present invention optionally comprises determining the variables associated with the sedation of a patient.

In a second aspect, the present invention relates to a device for determining intracranial pressure in a patient (hereinafter, device of the present invention) by means of the method of the present invention, which comprises means for determining the values of the spectral analysis and network variables for an electroencephalogram; and means for calculating the transfer function from said variables.

More particularly, the device of the present invention further comprises a microprocessor.

More particularly, the device of the present invention comprises means for recording the electroencephalographic and electrocardiographic signals.

More particularly, the device of the present invention comprises a supervised learning algorithm.

### DESCRIPTION OF THE FIGURES

FIG. 1 shows the continuous monitoring of 3 consecutive days of different measurements of the EEG, ICP and heart rate, in each of the 5-second time windows. The measurements derived from the EEG are: the density of links, “density”, the relative spectral power in the Delta, Theta, Alpha bands and spectral entropy. The measurements of the ICP and the differential thereof, “diff-ICP”, can also be observed, as well as the heart rate, estimated by means of an electrode responsible for measuring cardiac activity (V3 lead).

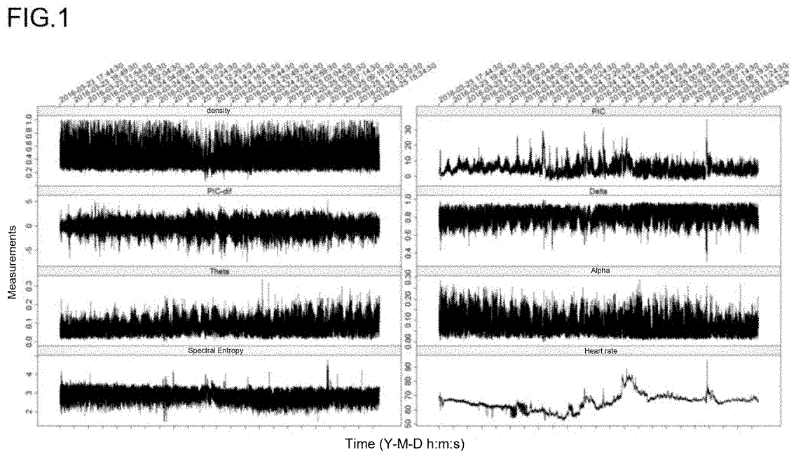

FIG. 2 shows estimates of the Granger causality (GC) between all the variables throughout one day of continuous monitoring. When the value of F, determined by Equation (2), for this pair of variables is not significant (P<0.05), a zero was put in that time window. Otherwise, the corresponding value has been graphed. The colour scale of these values is found to the right of the figure, the highest values being those in red. In order to make the graph clearer, all the F values greater than 5 have been made equal to 5. Since the non-significant values of F have been zeroed, they are blank in FIG. 2.

FIG. 3 shows the lag in cross-correlation between the different variables. It can be observed that there is no correlation with lag (or lead) between variables derived from the EEG, but it does exist between the variables from the EEG and the ICP (and the differential thereof). In most cases, it is clear that there is lead (coloured blue) in the maximum value of the correlation, this fact implying that the variables derived from the EEG have lag with respect to the ICP. According to the colour scale of the graph, on average this lag is between 5 and 10 steps. Given that these steps are actually 5-second time windows, this would correspond to a lag of 25 and 50 seconds.

FIG. 4 shows the percentages of Granger causality (GC) for the patient sample.

FIG. 5 shows a particular embodiment of the device of the present invention (7), comprising means for determining the values of the spectral analysis (8) and network (9) variables for an electroencephalogram; and means for calculating the transfer function (10) from said variables, and as optional components: a microprocessor (11), means for recording the electroencephalographic signal (12) and the electrocardiographic signal (13), a supervised learning algorithm (14) and a screen for seeing the data (15).

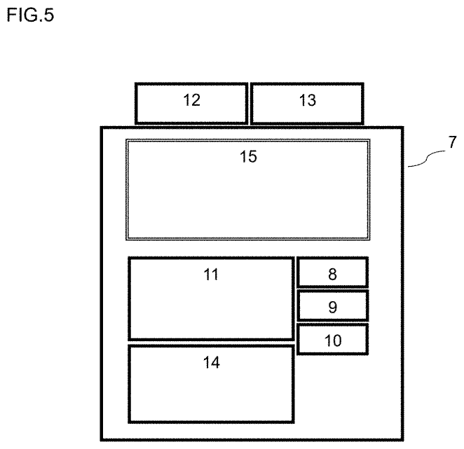

### DETAILED DESCRIPTION OF THE INVENTION

We have analysed the records of 18 patients (8 of whom are women) admitted to the ICU of the Hospital de la Princesa during the period from October 2015 to March 2017. All patients were monitored continuously with scalp EEG and ICP. The data analysis was performed retrospectively and always with the informed consent of the patients or their families. The research study was approved by the Ethics Committee of the Hospital de la Princesa. Inclusion criteria were as follows: patients of both sexes, over 18 years of age, having TBI or SAH, Glasgow scale less than 9, ICP monitoring. The exclusion criteria included: patients with a stay of less than one week, impossibility of continuous recording of EEG. The continuous monitoring of EEG was done by means of 19 scalp electrodes, mounted in a standard 10-20 electrode placement configuration. The records have been sampled at a frequency of 500 Hz and a monopolar assembly referencing the midline of the electrodes has always been used, meaning, (Fz+Cz+Pz)/3. The records were acquired continuously for a period (on average) of 5.2±2.3 days for each patient. In order to eliminate segments of records with artefacts from patient manipulation, interference with other equipment, etc., a video camera was installed which enables the patient to be monitored continuously.

Each EEG record has been divided into 5-second windows which, due to the fact that they are sampled at 500 Hz, correspond to windows with 2500 data points in each of the 19 channels. We have verified that in these 5-second windows the records have an acceptable weak stationarity and therefore we have calculated several measurements, both spectral and network ones. In particular, we have calculated the relative spectral power, meaning, the power in each band with respect to the total power, for each of the Delta, Theta, Alpha, Beta and Gamma bands, as well as the density of links that the interaction network has [Sanz-Garcia, A., Vega-Zelaya, L., Pastor, J., Sola, R. G., & Ortega, G. J. (2017). Towards Operational Definition of Postictal Stage: Spectral Entropy as a Marker of Seizure Ending. Entropy, 19(2), 81.]. For the purpose of calculating this last measurement, first we have calculated an estimator for the level of synchronisation existing between the activity recorded between each pair of electrodes. We have used the following statistics as synchronisation estimators: Pearson correlation, phase synchronisation, mutual information and coherence [Mezeiova K., Paluu M. (2012). Comparison of coherence and phase synchronization of the human sleep electroencephalogram. Clin Neurophysiol 123, 1821-1830]. We have obtained estimates of all these measurements at 5-second intervals, meaning, twelve values per minute, in non-overlapping windows.

Moreover, the ICP has also been continuously recorded [Kirkman, M. A., & Smith, M. (2013). Intracranial pressure monitoring, cerebral perfusion pressure estimation, and ICP/CPP-guided therapy: a standard of care or optional extra after brain injury?. British journal of anaesthesia, aet418; Kristiansson, H., Nissborg, E., Bartek, J., Andresen, M., Reinstrup, P., & Romner, B. (2013) Measuring Elevated Intracranial Pressure through Noninvasive Methods: A Review of the Literature. J Neurosurg Anesthesiol, 25 (4):372-85.] in those patients in whom, due to their pathology and particular condition, it has been indicated by neurosurgeons and intensive care specialists. In these cases, we have obtained continuous intracranial pressure records, measured by means of a Camino fibreoptic sensor [Martinez-Manas, R. M., Santamarta, D., de Campos, J. M., & Ferrer, E. (2000). Camino® intracranial pressure monitor: prospective study of accuracy and complications. Journal of Neurology, Neurosurgery & Psychiatry, 69(1), 82-86.] which, by means of a transducer that is located at the tip of an optical fibre and inserted into the parenchyma, allowing intracranial pressure values to be obtained thanks to changes in the intensity of light reflected in a mirror which is moved by the ICP. The ICP data was obtained and stored by means of a programme specifically designed for this purpose, NeuroPic. The sampling time of the ICP is approximately 2.9 seconds between successive values, although this can vary. In order to remove this variation in the sampling frequency and obtain “average” ICP data at times coinciding with those of the EEG time windows, we have re-sampled this signal with a sampling time of 5 seconds between consecutive data points. In this manner, we have ICP values for every minute, at 0 seconds, 5 seconds, 10 seconds, etc. until 12 ICP values are obtained for every minute. This temporal discretisation coincides with the one performed for the values of the time windows for the measurements calculated from the EEG records, as explained in the previous paragraph. It is worth mentioning that the ICP sensor tends to have drift in the initial calibration thereof, meaning that the zero of the initial pressure, calibrated with respect to the atmospheric pressure just before being inserted into the parenchyma, appears with values other than zero when taken off. In order to correct this deviation, we have removed the slope calculated between the initial zero and the final deviation value from the time series of the ICP. Generally, this value does not exceed ±2 mmHg.

We have also paired up the records of the measurements derived from the EEG and those of the ICP in order to obtain a new multivariate series with the measurements derived from the EEG, both spectral and network, and the average ICP values in those intervals. This is an important step for integrating the underlying dynamics for both types of records. We have also used the electrocardiograph (ECG) record obtained by means of the V3 lead and from it we have calculated the heart rate by measuring the distance between T waves, meaning T-T. Although the EEG and ICP records are continuous, in some situations one or both records are cut off, such as when the patient is moved to other services in the hospital.

Finally, in order to quantify the dependence that some time series may have on others, we have used the Granger Causality (GC) [Granger, C. W. (1969). Investigating causal relations by econometric models and cross-spectral methods. Econometrica: Journal of the Econometric Society, 424-438.], estimated as follows: given two time series, each of them Ndat; X=Xₖ, k=1; Ndat and Y=Y_{K}, K=1 Ndat, Granger causality basically examines whether the future values of one variable can be predicted by another. Numerically this can be evaluated by means of autoregressive models of order L that adjust each of the series such that:

$${x}_{k} = {\sum }_{i = 1}^{L} {a}_{i} {x}_{k - i} + {ɛ}_{x} \\ {x}_{k} = {\sum }_{i = 1}^{L} {a}_{i} {x}_{k - i} + {ɛ}_{x} + {\sum }_{i = 1}^{L} {b}_{i} {y}_{k - i} + {ɛ}_{y} \qquad ( 1 )$$

If the second prediction is better than the first one, it can be ensured that the past values of y affect the present values of x. The way to quantify “best” in a statistical sense is by means of a comparison between εₓ and ε_{y}, for example, using the statistic:

$${F}_{y \to x} = ln \frac{var ( {ɛ}_{x} )}{var ( {ɛ}_{y} )} \qquad ( 2 )$$

Such that F_{y→x} is non-negative and the larger F_{y→x} is, the better the fit in the combined model and therefore this implies a causality of y over x. The statistical significance of this equation can be assessed by means of Fisher's test.

$${F}_{y \to x} = \frac{\frac{{RSS}_{x} - {RSS}_{y}}{L}}{\frac{{RSS}_{y}}{{N}_{dat} - 2 L - 1}} \qquad ( 3 )$$

Where RSSx and RSSy are the residual sums of the squares of the x and y models, respectively. In our case, of course, x and y will be replaced by the variables ICP (or ICP-diff) and the measurements from the EEG.

To confirm the relationship between the ICP and the electroencephalographic activity, we calculated different measurements of dependence and/or correlation between pairs of variables. Regarding the ICP we used this variable as well as the differential thereof, meaning the first derivative thereof, since in this manner any trend existing in the time series is removed, thus making it more stationary.

We used the GC to study a possible dependence of one time series on another. To do so, we studied the GC for all the pairs that can be formed between the following variables: Delta, Theta, Alpha, density of links, Spectral Entropy, ICP and the differential of the ICP. We have used Equation (1) for each of the time series in each pair of variables and we calculated the statistic determined by Equation (2). We have calculated the statistical significance of this estimator by means of Fisher's test, given by Equation (3). The continuous monitoring of the EEG and the ICP enables us to have records with an extensive duration and therefore enables us to study the dynamics of the interactions between the variables. In order to quantify this, we have divided the total record into 30-minute time windows. In each of these time windows we have calculated the GC for each pair of variables. Taking into account that the data, both from the ICP and the EEG measurements, is sampled every 5 seconds (see explanation in the methodology section), we will have to make the GC evaluations over a length of x12=360 values (12 values per minute) in 30 minutes.

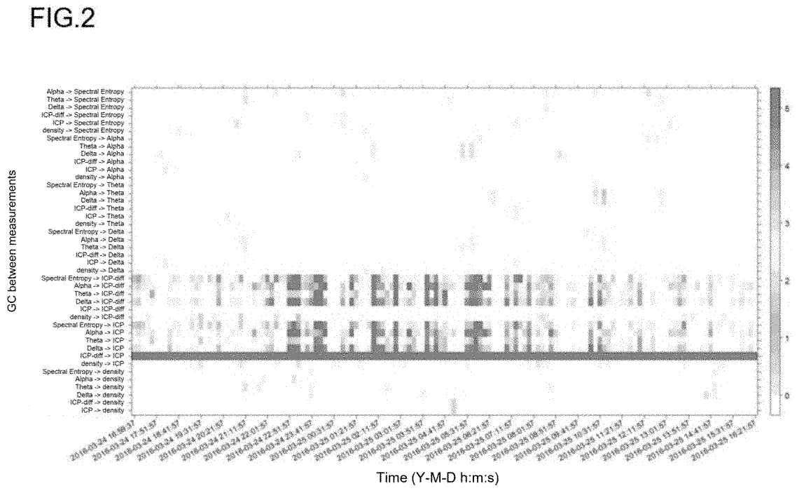

As shown in FIG. 2, there is a clear dependence on the ICP variable (and on ICP-diff, that is, the differential thereof), with respect to the measurements derived from the EEG, spectral entropy and the Delta, Theta and Alpha bands. It can also be seen that there is no such dependence in the opposite direction, or with respect to the network variable such as density of links (density). Although the dependence of the EEG variables on the ICP occurs throughout the entire record, in some areas it is more intense (for example, at about 11 PM on day 24) and less intense and even non-existent in other regions, such as for example at 9 AM on day 25.

Alternatively, we calculated the degree of correlation between all the pairs of variables in order to have an independent measurement of the degree of dependence existing between the variables studied. To do so, we calculated the cross-correlation between all the pairs of variables, in the same manner we used to study the GC. In order to study whether there is lead or lag in the correlation, which would imply causality, we have calculated the cross-correlation between all the pairs of variables with lag of up to a maximum of 30, meaning 30 displacements for one side and the other, between the two variables studied.

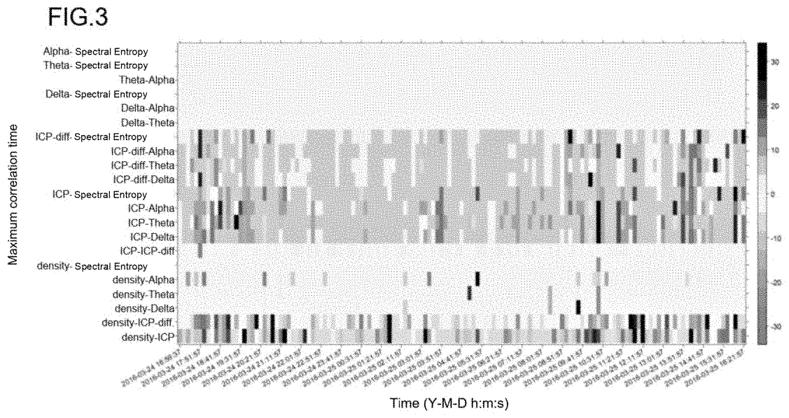

From the data shown in FIG. 3, it is concluded that there is no correlation with lag (or lead) between variables derived from the EEG, but it does exist between the variables from the EEG and the ICP (and the differential thereof). In most cases, it is clear that there is lead (coloured blue) in the maximum value of the correlation, this fact implying that the variables derived from the EEG have lag with respect to the ICP. According to the colour scale of the graph, on average this lag is between 5 and 10 steps. Given that these steps are actually 5-second time windows, this would correspond to a lag of 25 and 50 seconds. Curiously, there is also a certain correlation between the density of links and the ICP, although there is no pattern that clearly identifies what variable has lead compared to which.

According to the previous results, the dependence between the measurements of the EEG and the ICP for 18 patients admitted to the ICU of the Hospital de la Princesa was checked. In these cases, we have found that in the majority of patients there was a strong dependence between the variables of the EEG (the bands and spectral entropy) on the ICP. In the same manner the calculations were made in FIG. 2, we calculated the GC over the time in which the patients were monitored during their stay in the ICU, of both ICP and EEG. An example for one of the patients can be seen in table 1. We have calculated the “percentage” of time in which the GC is significant (P>0.05) during the entire monitoring process. This calculation has been made for different values of L, meaning the length of the autoregressive model in equation (1) which is somehow related to the lag existing between both variables.

TABLE 1
significant GC percentages for different variables of a patient.

| | Lag = 5 | Lag = 10 | Lag = 15 |
|---|---|---|---|
| Spectral entro −> icp | 60.87 | 57.39 | 49.57 |
| Spectral entro −> icp-diff | 60.87 | 57.39 | 50.43 |
| alpha −> icp | 52.17 | 46.09 | 43.48 |
| alpha −> icp-diff | 47.83 | 46.96 | 41.74 |
| delta −> icp | 57.39 | 53.91 | 49.57 |
| delta −> icp-diff | 53.91 | 53.91 | 46.09 |
| icp −> Spectral entro | 1.74 | 0.87 | 2.61 |
| icp-diff −> Spectral entro | 0.87 | 0.87 | 0.87 |
| icp −> alpha | 1.74 | 2.61 | 1.74 |
| icp-diff −> alpha | 0.00 | 1.74 | 0.00 |
| icp −> delta | 1.74 | 2.61 | 0.87 |
| icp-diff −> delta | 0.00 | 0.00 | 0.87 |

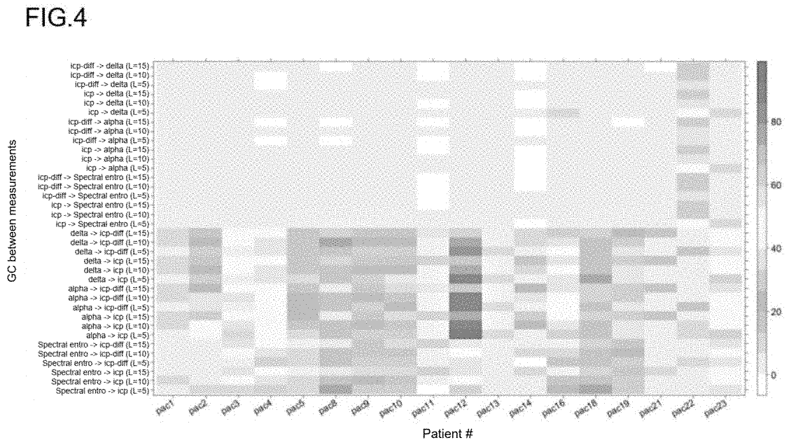

This same procedure has been performed in the set of 18 patients studied. FIG. 4 shows the percentages of the GC throughout all the records for each of the patients, and for three lag values, Lag=5, 10 and 15.

The first impression of the figure is that, except in a few cases (patients 13 and 22), the trend observed in the example in table 1 is repeated, meaning that the percentages of GC between the spectral variables of the EEGs are much greater than the inverse ones, which in most cases disappear. Second, it is notable that the percentages vary in the same patient according to the lag existing between both time series. For example, in patient 11, the percentage of causality between the variables of the EEG over the ICP is much greater when the lag is 15, compared to a lag of 5 or 10. This fact would imply that the lag or duration of the causality effect of one variable over another is dependent on the patient.

Finally, an automatic classification method, Support Vector Machines (SVM), was used to determine a model that would enable the increase in ICP to be inferred. SVM is a non-linear classification algorithm [Chang C. C., Lin C. J. (2011). LIBSVM: A library for support vector machines. ACM Trans Intell Syst Technol TIST, 2:27.]. SVM was used as follows: all the time windows of all the patients were grouped in order to construct a general model, two-thirds of the randomly-chosen windows were used to train the algorithm, each window was assigned an ICP value calculated by the invasive method, in addition to all the variables derived from the EEG; one third of the randomly-chosen windows were used to determine the efficiency of the model. By means of a confusion matrix, the prediction percentage of the model was determined, which reached 91% prediction, decreasing to 89% when subtracting chance.

Our work analysing simultaneous EEG and ICP records in patients hospitalised in the ICU of the Hospital de la Princesa shows the existence of a direct relationship between the dynamics of the ICP and certain variables calculated from the EEG records.

The data was calculated from a device (7) comprising means for determining the values of the spectral analysis (8) and network (9) variables for an electroencephalogram, as well as means for calculating the transfer function (10) from said variables. Optionally, it contained a microprocessor (11), means for recording the electroencephalographic signal (12) and electrocardiographic signal (13), a supervised learning algorithm (14) and a screen to see the data (15).

1. A method for determining changes in intracranial pressure using data obtained from an electroencephalogram recorded for a patient comprising:
a) determining the values of the spectral analysis and network variables for the electroencephalogram,
b) determining the endogenous variable X for a transfer function the exogenous variables of which are the values for the variables obtained in step a),
whereby changes in the endogenous variable X are indicative of changes in the value of the intracranial pressure.

2. The non-invasive method for determining intracranial pressure according to claim 1, wherein the spectral analysis variables for the electroencephalogram determined in step a) are selected from among: bands lower than delta (<1 Hz), delta (1-4 Hz), theta (4-7 Hz), alpha (8-12 Hz), beta (12-30 Hz), gamma (30-100 Hz), bands greater than gamma (>100 Hz), spectral entropy of all frequencies.

3. The non-invasive method for determining intracranial pressure according to claim 1, wherein the network analysis variables for the electroencephalogram determined in step a) are selected from among the variables obtained from the synchronisation between the different electrodes of the electroencephalogram: density of links, average clustering coefficient, mean path length and degree of each node.

4. The non-invasive method for determining intracranial pressure according to claim 1, wherein the value of the spectral analysis and network variables of stage a) is determined by the following steps:
I. calculating the power spectrum in the neurophysiological records from each electrode, by means of the Fourier transform;
II. calculating the relative power of each of the characteristic frequency bands of the electroencephalogram;
III. calculating the value of entropy and spectral entropy;
IV. calculating synchronisation measurements between all pairs of neurophysiological records using Pearson correlation, Mutual Information and Phase Synchronisation;
V. calculating the value of the density of links, average clustering coefficient, mean path length and degree of each node;
VI. estimating all the previous measurements in consecutive time windows.

5. The non-invasive method according to claim 1, comprising determining variables associated with the heart rate variability of an electrocardiogram recorded for a patient.

6. The non-invasive method according to claim 1, comprising determining the variables associated with the sedation of a patient.

7. A device for determining intracranial pressure in a patient by means of the method according to claim 1, comprising means for determining the values of the spectral analysis and network variables for an electroencephalogram; and means for calculating the transfer function from said variables.

8. The device according to claim 7, comprising a microprocessor.

9. The device according to claim 7, comprising means for recording the electroencephalographic signal and electrocardiographic signal.

10. The device according to claim 7, comprising a supervised learning algorithm.
